(19) United States
(12) Patent Application Publication
(10) Pub. No.: US 2008/0200564 A1
(43) Pub. Date: Aug. 21, 2008

(54) Copolymers Based on N-Vinylpyrrolidone and Branched Aliphatic Carbonxylic Acids, and Their Use as Solubilizers

(75) Inventors: Maximilian Angel, Schifferstadt (DE); Kathrin Meyer-Bohm, Mannheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/997,208

(22) PCT Filed: Jul. 21, 2006
(86) PCT No.: PCT/EP2006/064554

§ 371 Date: Jan. 29, 2008

(30) Foreign Application Priority Data
Jul. 29, 2005 (DE) 10 2005 036 328.8

(51) Int. Cl. A61K 47/32

(52) U.S. Cl. 514/772.6

(57) ABSTRACT

Copolymers comprising: (a) 60 to 99% by weight of at least one monomer selected from the group consisting of N-vinyllactams, N-vinylamides, and mixtures thereof; (1) 1 to 40% by weight of at least one monomer selected from the group consisting of vinyl esters of aliphatic branched C₈-C₃₀-carboxylic acids; (c) 0 to 30% by weight of vinyl acetate; and (d) 0 to 39% by weight of at least one additional free-radically copolymnerizable monomer; wherein the % by weight content of components (a), (b), (c) and (d) totals 100%, and with the proviso that the total amount of component (b) and component (c) combined is 1 to 40% by weight based on the copolymer, are described along with methods of using such copolymers to solubilize substances which are insoluble in water, substances which are only sparingly soluble in water, and combinations thereof.

The invention relates to copolymers based on N-vinyllactams or N-vinylamides and vinyl esters of branched aliphatic carboxylic acids, to the preparation thereof and to the use thereof as solubilizers of substances which are sparingly soluble in water. In addition, the invention relates to corresponding preparations for use on humans, animals and plants.

When producing homogeneous preparations of biologically active substances, the solubilization of hydrophobic substances, i.e. substances which are sparingly soluble in water, has achieved very great practical importance.

Solubilization is understood as meaning making substances which are insoluble or sparingly soluble in a certain solvent, in particular water, soluble through interface-active compounds, the solubilizers. Such solubilizers are able to convert poorly water-soluble or water-insoluble substances into clear, at most opalescent aqueous solutions without the chemical structure of the substances undergoing a change in the process (cf. Rbmpp Chemie Lexikon, 9^{th} edition, Vol. 5, p. 4203, Thieme Verlag, Stuttgart, 1992).

The prepared solubilizates are notable for the fact that the poorly water-soluble or water-insoluble substance is present in colloidally dissolved form in the molecular associates of the surface-active compounds which form in aqueous solution—the so-called micelles. The resulting solutions are stable single-phase systems which appear to be visually clear to opalescent and can be prepared without the input of energy.

Solubilizers can, for example, improve the appearance of cosmetic formulations and of food preparations by making the formulations transparent. Furthermore, in the case of pharmaceutical preparations, the bioavailability and thus the effect of medicaments can also be increased through the use of solubilizers.

The solubilizers used for pharmaceutical medicaments and cosmetic active ingredients are primarily surfactants such as ethoxylated (hydrogenated) castor oil, ethoxylated sorbitan fatty acid esters or ethoxylated hydroxystearic acid.

However, the hitherto used solubilizers described above have a number of applications-related disadvantages.

Thus, for example, their parenteral application is associated with a release of histamine and a drop in the blood pressure resulting therefrom (Lorenz et al., Agents and Actions, Vol. 12, 1/2, 1982).

The known solubilizers have only a small solubilizing effect for some sparingly soluble medicaments such as, for example, clotrimazole.

Interface-active compounds often have high hemolytic activity, which precludes an application in the field of pharmacy, particularly in substances administered parenterally.

A further desirable requirement of solubilizers is the ability to form so-called “solid solutions” with sparingly soluble substances. The term solid solutions refers to the state in which a substance is in molecularly disperse distribution in a solid matrix, for example a polymer matrix. Such solid solutions lead, for example when used in solid pharmaceutical administration forms of a sparingly soluble active ingredient, to an improved release of the active ingredient. An important requirement of such solid solutions is that they are stable even upon storage over a prolonged period, i.e. that the active ingredient should not crystallize out.

When forming solid solutions, besides the fundamental ability of the solubilizers to form solid solutions, the hygroscopicity of the solubilizers also plays an important role. Solubilizers which absorb too much water from the ambient air lead to deliquescence of the solid solution and to undesired crystallization of the active ingredients. Excessive hygroscopicity can also cause problems during processing to give administration forms.

U.S. Pat. No. 4,432,881 describes hydrophobically modified polyacrylic acid with a molecular weight between 200 000 and 5 000 000 which are obtained by copolymerization of acrylic acid with the corresponding N-alkylacrylamides or acrylates. The polymers obtained are used as dispersible hydrophobic thickeners.

U.S. Pat. No. 4,395,524 describes the copolymerization of hydrophilic components (e.g. acrylamide, acrylic acid, N-vinylpyrrolidone etc.) with N-alkylacrylamides. The polymers obtained in this way with a molecular weight of from 30 000 to 2 000 000 are used as thickeners, sedimentation stabilizers or dispersants.

EP-A-0 268 164 describes the use of copolymers of monoolefinically unsaturated acids and alkyl esters of monoolefinically unsaturated acids for stabilizing O/W emulsions.

EP-A 876 819 describes the use of copolymers of at least 60% by weight of N-vinylpyrrolidone and amides or esters with long-chain alkyl groups.

EP-A 948 957 describes the use of copolymers of monoethylenically unsaturated carboxylic acids such as, for example, acrylic acid and hydrophobically modified comonomers such as, for example, N-alkyl- or N,N-dialkylamides of unsaturated carboxylic acids with C₈-C₃₀-alkyl radicals.

The polymeric solubilizers known to date have the disadvantages that they either do not form stable solid solutions or are too hygroscopic. Furthermore, they still leave room for improvements with regard to solubilization in aqueous systems.

The object was to provide novel solubilizers for pharmaceutical, cosmetic, food and agrotechnical applications.

This object was achieved by copolymers comprising
a) 60 to 99% by weight of at least one monomer chosen from the group of N-vinyllactams and N-vinylamides,
b) 1 to 40% by weight of at least one monomer chosen from the group of vinyl esters of aliphatic branched C₈-C₃₀-carboxylic acids,
c) 0 to 30% by weight of vinyl acetate,
d) 0 to 39% by weight of at least one further free-radically copolymerizable monomer, where the % by weight data for the individual components add up to 100% by weight, and with the proviso that the sum of the amounts of b) and c) is 1 to 40% by weight of the total amount.

In addition, the invention relates to the use thereof as solubilizers for substances which are sparingly soluble in water, and also to corresponding preparations.

Suitable monomers a) are N-vinyllactams, such as N-vinylpyrrolidone, N-vinylpiperidone and N-vinylcaprolactam, or N-vinylamides, such as N-methyl-N-vinylacetamide, N-vinylacetamide and N-vinylfornamide.

Preferred monomers a) are N-vinylpyrrolidone and N-vinylcaprolactam.

The proportion of the monomer a) in the copolymer is preferably in the range from 70 to 95% by weight, particularly preferably in the range from 74 to 94% by weight.

According to the invention, suitable hydrophobic components b) are vinyl esters of aliphatic branched, in particular saturated branched, C₈-C₃₀-carboxylic acids. Of suitability in this connection are, in particular, the vinyl esters of the so-called versatic® acids having at least 8 carbon atoms. The versatic acids are heavily branched saturated monocarboxylic acids with tertiary carboxyl groups, where the a-branching point carries at least one methyl group and the numerical datum refers to the total number of carbon atoms, meaning that versatic 8 is, for example, 2,2-dimethylhexanoic acid. Suitable acids are also, for example, 2,2-dimethylheptanoic acid, 2-ethyl-2-methyl-heptanoic acid, 2,2-dimethyloctanoic acid, 2-ethyl-2-methyloctanoic acid or 2,2-dimethylnonanoic acid, preference being given to vinyl esters of versatic 9 and versatic 10 acids Such vinyl esters of versatic acids are commercially available.

The proportion of the hydrophobic monomer building blocks b) in the copolymer is preferably in the range from 5 to 30% by weight, particularly preferably 10 to 20% by weight.

As monomer c), vinyl acetate is used in amounts of up to 30% by weight. The sum of the amounts of monomers b) and c) is preferably 8 to 30% by weight, particularly preferably 10 to 30% by weight.

The sum of the components a) to c) is particularly preferably 100% by weight.

In addition, the copolymers can comprise the following free-radically copolymerizable monomers d):

N—C₈-C₃₀-alkyl- or N,N-C₈-C₃₀-dialkyl-substituted amides of monoethylenically unsaturated C₃-C₈-carboxylic acids, where the alkyl radicals are straight-chain or branched aliphatic or cycloaliphatic alkyl radicals having 8 to 30, preferably 8 to 18, carbon atoms. Suitable monoethylenically unsaturated carboxylic acids having 3 to 8 carbon atoms here are acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid, preferably acrylic acid, methacrylic acid, maleic acid or mixtures of the specified carboxylic acids.

Preferred amidated comonomers are, for example, N-stearylacrylamide, N-stearyl-methacrylamide, N-(1-methyl)undecylacrylamide, N-(1-methyl)undecylmethacrylamide, N-dodecylacrylamide, N-dodecylmethacrylamide, N-octylacrylamide, N-octylmethacrylamide, N,N-dioctylacrylamide, N,N-dioctylmethacrylamide, N-cetylacrylamide, N-cetylmethacrylamide, N-dodecylacrylamide, N-dodecylmethacrylamide, N-myristylacrylamide, N-myristylmethacrylamide, N-(2-ethyl)hexylacrylamide, N-(2-ethyl)hexylmethacrylamide.

In the case of maleic anhydride as comonomer, this can be reacted in a polyrner-analogous manner with N-alkylamines by ring opening to give the corresponding amides.

Further comonomers d) which can be used are monoethylenically unsaturated C₃-C₈-carboxylic esters with a C₈-C₃₀-alcohol, preferably a C₈-C₁₈-alcohol.

Of particular importance in this connection are the acrylic and methacrylic esters with fatty alcohols with a chain length of from 8 to 18 carbon atoms, where the alkyl radicals may be branched or unbranched.

In particular, mention may be made here of: octyl acrylate, 2-ethylhexyl acrylate, nonyl acrylate, decyl acrylate, lauryl acrylate, myristyl acrylate, cetyl acrylate, stearyl acrylate, oleyl acrylate, behenyl acrylate, octyl methacrylate, 2-ethylhexyl methacrylate, nonyl methacrylate, decyl methacrylate, lauryl methacrylate, myristyl methacrylate, cetyl methacrylate, stearyl methacrylate, oleyl methacrylate, behenyl methacrylate, tert-butylcyclohexyl acrylate.

As further additional component d), vinyl esters of long-chain aliphatic, saturated or unsaturated, unbranched C₈-C₃₀-carboxylic acids, such as, for example, caprylic acid, capric acid, lauric acid, myristic acid, palmitic acid, stearic acid, arachidic acid, behenic acid, lignoceric acid, cerotinic acid, and melissic acid can be used.

In addition, as monomers d), C₈-C₃₀-alkyl vinyl ethers, preferably C₈-C₁₈-alkyl vinyl ethers, can be copolymerized. Preferred alkyl radicals of the vinyl ethers which may be mentioned are branched or unbranched C₈-C₁₈-alkyl chains, such as, for example, n-octyl, 2-ethylhexyl, n-nonyl, n-decyl, n-undecyl, n-dodecyl, n-tridecyl, n-tetradecyl, n-pentadecyl, n-hexadecyl, n-heptadecyl and n-octadecyl.

Suitable additional free-radically copolymerizable monomers d) are:

monoethylenically unsaturated carboxylic acids having 3 to 8 carbon atoms, such as, for example, acrylic acid, methacrylic acid, dimethacrylic acid, ethacrylic acid, maleic acid, citraconic acid, methylenemalonic acid, allylacetic acid, crotonic acid, fumaric acid, mesaconic acid and itaconic acid.

From this group of monomers, preference is given to using acrylic acid, methacrylic acid or mixtures of the specified carboxylic acids.

The monoethylenically unsaturated carboxylic acids can be used in the copolymerization as free acid, as anhydrides, and also in partially or completely neutralized form.

For the neutralization of the abovementioned carboxylic acids, preference is given to using alkali metal or alkaline earth metal bases, ammonia or amines, preferably sodium hydroxide solution, potassium hydroxide solution, sodium carbonate, potassium carbonate, sodium hydrogen carbonate, magnesium oxide, calcium; hydroxide, calcium oxide, gaseous or aqueous ammonia, triethylamine, ethanolamine, diethanolamine, triethanolamine, morpholine, diethylenetriamine or tetraethylenepentamine.

Further suitable comonomers d) to be used for the polymerization are, for example, monoethylenically unsaturated C₃-C₈-carboxylic esters of short-chain C₁-C₄-alcohols or nitriles in amounts of from 0 to 5 mol %.

Examples which may be mentioned are: methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, N-dimethylacrylamide, N-tert-butylacrylamide, acrylonitrile, methacrylonitrile, dimethylaminoethyl acrylate, diethylaminoethyl acrylate, dimethylaminoethyl methacrylate, diethylaminoethyl methacrylate, and the salts of the last-mentioned monomers with carboxylic acids or mineral acids, and also the quaternized products.

Further suitable monomers d) are, for example:
acrylamidoglycolic acid, vinylsulfonic acid, allylsulfonic acid, methallylsulfonic acid, styrenesulfonic acid, (3-sulfopropyl) acrylate, (3-sulfopropyl) methacrylate and acrylamidomethylpropanesulfonic acid;
monomers comprising phosphonic acid groups, such as vinyl phosphonic acid, allylphosphonic acid and acrylamidomethanepropane phosphonic acid.

It is of course also possible to use mixtures of the specified monomers.

Preferred monomers d) are acrylic acid, methacrylic acid, methyl methacrylate, ethyl acrylate, vinyl laurate, stearyl methacrylate and lauryl acrylate.

The proportion of the monomer building blocks d) in the copolymer is preferably in the range from 0 to 5% by weight, particularly preferably it is 0% by weight.

The copolymers used according to the invention can have K values in accordance with Fikentscher, measured at 1% strength by weight in ethanol, of from 10 to 200, preferably 15 to 100, particularly preferably 20 to 50.

The copolymers are prepared by free-radically polymerizing the corresponding monomers.

The preparation is carried out by known processes, e.g. solution polymerization, precipitation polymerization or by inverse suspension polymerization using compounds which form free radicals under the polymerization conditions.

The polymerization temperatures are usually in the range from 30 to 200° C., preferably 40 to 110° C. Suitable initiators are, for example, azo and peroxy compounds, and the customary redox initiator systems, such as combinations of hydrogen peroxide and reducing compounds, e.g. sodium sulfite, sodium bisulfite, sodium formaldehyde sulfoxylate and hydrazine.

The reaction medium used is any customary solvent in which the monomers are soluble. Preference is given to using water or alcoholic solvents, such as, for example, methanol, ethanol, n-propanol or isopropanol or mixtures of such alcohols with water.

In order to ensure that the reaction lead to homogeneous products, it is advantageous to supply the monomers and the starter separately to the reaction solution. This can take place, for example, in the form of separate feeds for the individual reactants.

The polymerization can also be carried out in the presence of customary regulators if relatively low molecular weights are to be established.

The solids content of the organic solution obtained is usually 20 to 60% by weight, in particular 25 to 40% by weight.

A nonaqueous solvent used for the polymerization can then be removed by means of steam distillation and be replaced by water.

The aqueous solutions of the copolymers can, by various drying processes such as, for example, spray-drying, fluidized spray drying, drum drying or freeze-drying, be converted into powder form, from which an aqueous solution can again be prepared by redispersion in water.

Applications:

The copolymers to be used according to the invention can in principle be used in all fields where substances which are insoluble or only sparingly soluble in water are either to be used in aqueous preparations or are to develop their effect in an aqueous medium. Accordingly, the copolymers are used as solubilizers of substances which are sparingly soluble in water, in particular biologically active substances.

According to the invention, the term “sparingly soluble in water” also comprises virtually insoluble substances and means that for a solution of the substance in water at 20° C., at least 30 to 100 g of water per g of substance are required. In the case of virtually insoluble substances, at least 10 000 g of water per g of substance are required.

For the purposes of the present invention, biologically active substances which are sparingly soluble in water are understood as meaning pharmaceutical active ingredients for humans and animals, cosmetic or agrochemical active ingredients or food supplements or dietetic active ingredients.

In addition, suitable sparingly soluble substances to be solubilized are also dyes, such as inorganic or organic pigments.

By virtue of the present invention, amphiphilic compounds in particular for use as solubilizers for pharmaceutical and cosmetic preparations and also for food preparations are provided. They have the property of solubilizing sparingly soluble active ingredients in the field of pharmacy and cosmetics, sparingly soluble food supplements, for example vitamins and carotenoids, but also sparingly soluble active ingredients for use in crop protection compositions, and also veterinary medicine active ingredients.

Surprisingly, in the case of the claimed compounds, a good solubilization ability for pharmaceutical and cosmetic active ingredients has been found. In addition, with the claimed compounds, uses are obtained which are notable for a very low hemolysis rate, side-effect-free compatibility following parenteral, oral and topical application to skin and mucosa. In particular, the compounds have no secondary reactions due to interactions with blood corpuscle membranes. Following parenteral application, no or only a slight release of histamine takes place. The molecular weight of the solubilizers can, if desired, be adjusted so that the solubilizers pass through the kidneys.

Solubilizers for Cosmetics:

According to the invention, the copolymers can be used as solubilizers in cosmetic formulations. For example, they are suitable as solubilizers for cosmetic oils. They have a good solubilizing ability for fats and oils, such as peanut oil, jojoba oil, coconut oil, almond oil, olive oil, palm oil, castor oil, soybean oil or wheatgerm oil or for essential oils, such as dwarf pine oil, lavender oil, rosemary oil, spruce needle oil, pine needle oil, eucalyptus oil, peppermint oil, sage oil, bergamot oil, terpentine oil, Melissa oil, sage oil, juniper oil, lemon oil, anise oil, cardamom oil; peppermint oil, camphor oil etc. or for mixtures of these oils.

In addition, the polymers according to the invention can be used as solubilizers for UV absorbers which are insoluble or sparingly soluble in water, such as, for example, 2-hydroxy-4-methoxybenzophenone (Uvinul® M 40, BASF), 2,2′,4,4′-tetrahydroxybenzophenone (Uvinul® D 50), 2,2′-dihydroxy-4,4′-dimethoxybenzophenone (Uvinul® D49), 2,4-dihydroxybenzophenone (Uvinul® 400), 2′-ethylhexyl 2-cyano-3,3-diphenylacrylate (Gvinul® N 539), 2,4,6-trianilino-p-(carbo-2′-ethylhexyl-1′Boxy)-1,3,5-triazine (Uvinul® T 150), 3-(4-methoxybenzylidene)camphor (Eusolex® 6300, Merck), 2-ethylhexyl N,N-dimethyl-4-aminobenzoate (Eusolex® 6007), 3,3,5-trimethylcyclohexyl salicylate, 4-isopropyldibenzoylmethane (Eusolex® 8020), 2-ethylhexyl p-methoxycinnamate and 2-isoamyl p-methoxycinnamate, and mixtures thereof.

The present invention therefore also provides cosmetic preparations which comprise at least one of the copolymers according to the invention of the composition specified at the start as solubilizers. Preference is given to those preparations which, besides the solubilizer, comprise one or more sparingly soluble cosmetic active ingredients, for example the abovementioned oils or UV absorbers.

These formulations are solubilizates based on water or water/alcohol. The solubilizers according to the invention are used in the ratio from 0.2:1 to 20:1, preferably 1:1 to 15:1, particularly preferably 2:1 to 12:1 relative to the sparingly soluble cosmetic active ingredient. The content of solubilizer according to the invention in the cosmetic preparation is, depending on the active ingredient, in the range from 1 to 50% by weight, preferably 3 to 40% by weight, particularly preferably 5 to 30% by weight.

In addition, further auxiliaries can be added to this formulation, for example nonionic, cationic or anionic surfactants, such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc. Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath additive preparations such as bath oils, aftershaves, face tonics, hair tonics, eau de cologne, eau de toilette and in sunscreen compositions. In addition, they are used in the area of oral care, for example in toothpastes, mouthwashes or mouth creams.

Description of the Solubilization Method:

In the preparation of the solubilizates for cosmetic formulations, the copolymers according to the invention can be used as 100% strength substance or preferably as aqueous solution.

Usually, the solubilizer is dissolved in water and vigorously mixed with the sparingly soluble cosmetic active ingredient to be used in each case.

However, it is also possible to vigorously mix the solubilizer with the sparingly soluble cosmetic active ingredient to be used in each case and then to add demineralized water with continuous stirring.

Solubilizers for Pharmaceutical Applications:

The claimed copolymers are likewise suitable for use as solubilizer in pharmaceutical preparations of any type which are notable for the fact that they can comprise one or more medicaments which are insoluble or sparingly soluble in water, and also vitamins and/or carotenoids. In particular, these are aqueous solutions or solubilizates for oral application or particularly preferably for parenteral application, such as, for example, injection solutions for intravenous, intramuscular or subcutaneous or intraperitoneal application.

Furthermore, the claimed copolymers are suitable for use in oral administration forms such as tablets, capsules, powders, solutions. Here, they can make the sparingly soluble medicament available with increased bioavailability.

In the case of parenteral application, it is also possible to use emulsions, for example fatty emulsions, besides solubilizate. The claimed copolymers are also suitable for this purpose, in order to process a sparingly soluble medicament.

Pharmaceutical formulations of the abovementioned kind can be obtained by processing the claimed copolymers with pharmaceutical active ingredients by conventional methods and with the use of known and novel active ingredients.

The use according to the invention can additionally comprise pharmaceutical auxiliaries and/or diluents. Cosolvents, stabilizers, preservatives are especially mentioned as auxiliaries.

The pharmaceutical active ingredients used are substances which are insoluble or slightly soluble in water According to DAB 9 (German Pharmacopoeia), the solubility of pharmaceutical active ingredients is categorized as follows: slightly soluble (soluble in 30 to 100 parts of solvent); sparingly soluble (soluble in 100 to 1000 parts of solvent); virtually insoluble (soluble in more than 10 000 parts of solvent). The active ingredients can here come from any area of indication.

Examples which may be mentioned here are benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, antiParkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiastbmatics, broncholytics, beta-receptor blockers, calcium antagonists, ACE inhibitors, antiarteriosclerotics, anti-inflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

One possible preparation variant is to dissolve the solubilizer in the aqueous phase, if appropriate with gentle heating and then to dissolve the active ingredient in the aqueous solubilizer solution. The simultaneous dissolution of solubilizer and active ingredient in the aqueous phase is likewise possible.

The use of the copolymers according to the invention as solubilizers can, for example, also be carried out by dispersing the active ingredient in the solubilizer, if appropriate with heating, and mixing it with water with stirring.

The invention thus also provides pharmaceutical preparations which comprise at least one of the copolymers according to the invention as solubilizer. Preference is given to those preparations which, besides the solubilizer, comprise a pharmaceutical active ingredient which is insoluble or sparingly soluble in water, for example from the abovementioned areas of indication.

Of the abovementioned pharmaceutical preparations, particular preference is given to those which are parenterally applicable formulations.

The content of solubilizer according to the invention in the pharmaceutical preparation is, depending on the active ingredient, in the range from 1 to 50% by weight, preferably 3 to 40% by weight, particularly preferably 5 to 30% by weight.

Solubilizers for Food Preparations:

Besides the use in cosmetics and pharmacy, the copolymers according to the invention are also suitable as solubilizers in the food sector for nutrients, auxiliaries or additives which are insoluble or sparingly soluble in water, such as, for example, fat-soluble vitamins or carotenoids. Examples which may be mentioned are clear drinks colored with carotenoids.

Solubilizers for Crop Protection Preparations:

The use of the copolymers according to the invention as solubilizers in agrochemistry can comprise, inter alia, formulations which comprise pesticides, herbicides, fungicides or insecticides, especially also those preparations of crop protection compositions which are used as spray mixtures or pouring mixtures.

The water-soluble copolymers according to the invention are notable for a particularly good solubilizing effect. They are also exceptionally suitable for producing stable solid solutions.

In the examples below, the preparation and use of the copolymers according to the invention is illustrated in more detail.

### EXAMPLES

The abbreviation VEOVA is used in the examples below for vinyl esters of versatic acids. The number after the abbreviation refers to the number of carbon atoms. The monomers are commercially available.

To prepare the polymers, the following apparatus was used:

21 HWS pot with water bath, anchor stirrer and thermometer. The HWS pot had connectors for 3 feeds, a reflux condenser and an inlet tube for introducing nitrogen or steam.

### Example 1

### Preparation of copolymers of N-vinylpyrrolidone/vinyl acetate/VEOVA 9 (weight ratio 70/15/15)

| | Amount g | Substance |
|---|---|---|
| Initial charge | 12.0 | Isopropanol |
| | 70.0 | Part of feed 1 |
| | 1.0 | Part of feed 2 |
| Feed 1 | 222.0 | Isopropanol |
| | 320.0 | Vinylpyrrolidone |
| | 75.0 | VEOVA 9 |
| | 75.0 | Vinyl acetate |
| Feed 2 | 85.4 | Isopropanol |
| | 4.5 | Tertiary-butyl perpivalate, 75% |
| | | strength |
| Feed 3 | 25.6 | Isopropanol |
| | 30.0 | Vinylpyrrolidone |

In the apparatus, 12 g of isopropanol and 70 g of feed 1 were mixed in the order given at 180 rpm. A gentle stream of nitrogen was passed continuously through the apparatus. At the same time, the system was heated to an internal temperature of 70° C. At 68° C., part of feed 2 (1.0 g) was added and the mixture was polymerized for 10 minutes.

Feed 1 and feed 2 were then started. Feed 1 was metered in over 4 hours. 37 g of feed 2 were metered in over 5 hours. When feed 1 was complete, feed 3 was metered in over 1 hour. When part of feed 2 had been added, the mixture was after-polymerized at 70° C. for a farther hour. The mixture was then heated to an internal temperature of 75° C. In parallel to the heating operation, feed 2 (residual amount 52 g) was started and metered in over 2 hours. At the end of feed 2, the mixture was after-polymerized at 75° C. for a further 2 hours.

400 g of completely demineralized water were then added and steam was passed into the polymer solution for 3 hours.

This gave a yellowish, viscous solution with a solids content of 32% by weight. The K value was 33 (measured at 1% strength by weight in ethanol).

### Example 2

### Preparation of copolymers of N-vinylpyrrolidone/VEOVA 9 (weight ratio 80/20)

| | Amount g | Substance |
|---|---|---|
| Initial charge | 12.0 | Isopropanol |
| | 78.0 | Part of feed 1 |
| | 5.0 | Part of feed 2 |
| Feed 1 | 222.0 | Isopropanol |
| | 400.0 | Vinylpyrrolidone |
| | 100.0 | VEOVA 9 |
| Feed 2 | 100.0 | Isopropanol |
| | 4.5 | Tertiary-butyl perpivalate, 75% |
| | | strength |

In the apparatus, 12 g of isopropanol and 78 g of feed 1 were mixed in the order given at 180 rpm. A gentle stream of nitrogen was passed continuously through the apparatus. At the same time, the system was heated to an internal temperature of 75° C. At 73° C., part of feed 2 (5.0 g) was added and the mixture was polymerized for 10 minutes.

Feed 1 and 2 were then started. Feed 1 was metered in over 4 hours, and feed 2 was metered in over 6 hours. The mixture was then after-polymerized for a further two hours.

400 g of completely demineralized water were then added and steam was passed into the polymer solution for about 3 hours.

This gives a yellowish, viscous solution with a solids content of 27% by weight. The K value is 35 (measured at 1% strength in ethanol).

### Example 3

### Preparation of copolymers of N-vinylpyrrolidone/VEOVA 9 (weight ratio 90/10)

| | Amount g | Substance |
|---|---|---|
| Initial charge | 12.0 | Isopropanol |
| | 78.0 | Part of feed 1 |
| | 5.0 | Part of feed 2 |
| Feed 1 | 222.0 | Isopropanol |
| | 450.0 | Vinylpyrrolidone |
| | 50.0 | VEOVA 9 |
| Feed 2 | 100.0 | Isopropanol |
| | 4.5 | Tertiary-butyl perpivalate, 75% |
| | | strength |

In the apparatus, 12 g of isopropanol and 78 g of feed 1 were mixed in the order given at 180 rpm. A gentle stream of nitrogen was passed continuously through the apparatus. At the same time, the system was heated to an internal temperature of 75° C. At 73° C., part of feed 2 (5.0 g) was added and the mixture was polymerized for 10 minutes.

Feed 1 and 2 were then started. Feed 1 was metered in over 4 hours, feed 2 was metered in over 6 hours. The mixture was then after-polymerized for a further two hours.

400 g of completely demineralized water were then added and steam was introduced into the polymer solution for about 3 hours.

This gives a yellowish, viscous solution with a solids content of 34% by weight.

The K value was 35 (measured at 1% strength by weight in ethanol).

### Example 4

### Preparation of copolymers of N-vinylpyrrolidone/N-vinylcaprolactam/VEOVA 10 (weight ratio 70/20/10)

| | Amount g | Substance |
|---|---|---|
| Initial charge | 12.0 | Isopropanol |
| | 70.0 | Part of feed 1 |
| | 1.0 | Part of feed 2 |
| Feed 1 | 222.0 | Isopropanol |
| | 320.0 | Vinylpyrrolidone |
| | 75.0 | VEOVA 9 |
| | 75.0 | Vinyl acetate |
| Feed 2 | 85.4 | Isopropanol |
| | 4.5 | Tertiary-butyl perpivalate, 75% |
| | | strength |
| Feed 3 | 25.6 | Isopropanol |
| | 30.0 | Vinylpyrrolidone |

In the apparatus, 12 g of isopropanol and 70 g of feed 1 were mixed in the order given at 180 rpm. A gentle stream of nitrogen was passed continuously through the apparatus. At the same time, the system was heated to an internal temperature of 70° C. At 68° C., part of feed 2 (1.0 g) was added and the mixture was polymerized for 10 minutes.

Feed 1 and 2 were then started. Feed 1 was metered in over 4 hours. 37 g of feed 2 were metered in over 5 hours. At the end of feed 1, feed 3 was metered in over 1 hour. When the part of feed 2 was complete, the mixture was after-polymerized at 70° C. for a further hour. The mixture was then heated to an internal temperature of 75° C. In parallel to the heating operation, feed 2 (remaining amount 52 g) was started and metered in over 2 hours. At the end of feed 2, the mixture was after-polymerized at 75° C. for a further 2 hours.

400 g of completely demineralized water were then added and steam was passed into the polymer solution for about 3 hours.

This gave a yellowish, viscous solution with a solids content of 32% by weight.

The K value was 47 (measured at 1% strength by weight in ethanol).

Tertiary-butyl perpivalate: 75% strength by weight active in aliphatics mixture, TBPPI-75-AL from Degussa, 82049 Pullach/Germany

Table of Example Experiments

| | Monomer | Monomer | Monomer | Monomer | Monomer |
|---|---|---|---|---|---|
| | a) | a) | b) | c) | d) |
| Example 1 | 70 VP | | 15 | 15 VAc | |
| | | | VEOVA-9 | | |
| Example 2 | 80 VP | | 20 | | |
| | | | VEOVA-9 | | |
| Example 3 | 90 VP | | 10 | | |
| | | | VEOVA-9 | | |
| Example 4 | 70 VP | 20 VCap | 10 | | |
| | | | VEOVA-10 | | |
| Example 5 | 50 VP | 20 VCap | 10 | 20 VAc | |
| | | | VEOVA-9 | | |
| Example 6 | 40 VP | 40 VCap | 10 | 10 VAc | |
| | | | VEOVA-9 | | |
| Example 7 | 50 VP | 30 VIMA | 20 | | |
| | | | VEOVA-9 | | |
| Example 8 | 92 VP | | 8 | | |
| | | | VEOVA-10 | | |
| Example 9 | 85 VP | | 10 | | 5 LA |
| | | | VEOVA-9 | | |

The polymers according to Examples 5 to 9 are prepared analogously to Examples 1 to 4.

All of the numerical data for the composition in % by weight.

| | |
|---|---|
| VP | N-Vinylpyrrolidone |
| VAc | Vinyl acetate |
| VEOVA-9 | Vinyl versatic acid 9 |
| VEOVA-10 | Vinyl versatic acid 10 |
| VCap | Vinylcaprolactam |
| VIMA | N-Vinyl-N-methylacetamide |
| LA | Lauryl acrylate |

Preparation of Solid Solutions: General Procedure

To prepare the polymer/active ingredient mixture, the active ingredient and the polymer were weighed into a suitable glass vessel in the weight ratio 1:1 (in each case 2 g) and then 16 ml of dimethylformamide were added as solvent. The mixture was stirred at 20° C. for 24 hours on a magnetic stirrer. The solution was then drawn out using a 120 μm doctor knife on a glass plate. This was dried in the fume cupboard at RT for 0.5 hours and then dried in the drying cabinet at 50° C. and 10 mbar for a further 0.5 hours in order to remove all of the solvent. The samples were then assessed visually. If the active ingredient did not crystallize out after 7 days, a stable solid solution had formed.

| Copolymer | | | |
|---|---|---|---|
| according to | Carbamazepine | Estradiol | Clotrimazol |
| Example 1 | solid solution | solid solution | solid solution |
| Example 2 | solid solution | solid solution | solid solution |
| Example 3 | solid solution | solid solution | solid solution |
| Example 4 | solid solution | solid solution | solid solution |
| Example 5 | solid solution | solid solution | solid solution |

Preparation of Solubilizates

2 g of the copolymer were weighed into a beaker. Then, one medicament in each case was weighed into the mixture as follows in order to obtain a supersaturated solution. (If the weighed-in mass dissolved in the medium, the initial weight was increased until a sediment formed).

| Active | Initial weight |
|---|---|
| ingredient | [g] |
| 17-β-Estradiol | 0.25 |
| Piroxicam | 0.25 |
| Carbamazepine | 0.35 |

Phosphate buffer pH 7.0 was then added until solubilizer and phosphate buffer were present in the weight ratio 1:9. Using a magnetic stirrer, this mixture was stirred at 20° C. for 72 hours. There then followed a resting time of at least one hour. Following filtration of the mixture, it was measured photometrically and the content of active ingredient was determined.

Results: content of solubilized active ingredient [g/100 ml]

| Copolymer according | | | |
|---|---|---|---|
| to | Carbamazepine | Estradiol | Piroxicam |
| Example 1 | 0.21 | 0.07 | 0.53 |
| Example 2 | 0.17 | 0.08 | 0.54 |
| Example 3 | 0.19 | 0.04 | 0.48 |
| Example 4 | 0.21 | 0.09 | 0.50 |
| Example 5 | 0.30 | 0.07 | 0.52 |

1-29. (canceled)

30. A method comprising:
providing a substance selected from the group consisting of substances which are insoluble in water, substances which are only sparingly soluble in water, and combinations thereof; and
combining the substance and a solubilizing amount of a copolymer; wherein the copolymer comprises:
(a) 60 to 99% by weight of at least one monomer selected from the group consisting of N-vinyllactamis, N-vinylamides, and mixtures thereof;
(b) 1 to 40% by weight of at least one monomer selected from the group consisting of vinyl esters of aliphatic branched C₈-C₃₀-carboxylic acids;
(c) 0 to 30% by weight of vinyl acetate; and
(d) 0 to 39% by weight of at least one additional free-radically copolymerizable monomer;
wherein the % by weight content of components (a), (b), (c) and (d) totals 100%, and with the proviso that the total amount of component (b) and component (c) combined is 1 to 40% by weight based on the copolymer.

31. The method according to claim 30, wherein the copolymer comprises:
(a) 70 to 95% by weight of the at least one monomer selected from the group consisting of N-vinyllactams, N-vinylamides, and mixtures thereof;
(b) 5 to 30% by weight of the at least one monomer selected from the group consisting of vinyl esters of aliphatic branched C₈-C₃₀-carboxylic acids;
(c) 0 to 25% by weight of vinyl acetate; and
(d) 0 to 25% by weight of the at least one additional free-radically copolymerizable monomer.

32. The method according to claim 30, wherein the copolymer has a K value of 10 to 200.

33. The method according to claim 30, wherein the at least one monomer selected from the group consisting of N-vinyllactams, N-vinylamides, and mixtures thereof comprises a compound selected from the group consisting of N-vinylpyrrolidone and N-vinylcaprolactam.

34. The method according to claim 30, wherein the at least one monomer selected from the group consisting of vinyl esters of aliphatic branched C₈-C₃₀-carboxylic acids comprises a vinyl ester of a versatic acid.

35. The method according to claim 30, wherein the at least one monomer selected from the group consisting of vinyl esters of aliphatic branched CS-C₃₀-carboxylic acids comprises a vinyl ester of a C₉-C₁₀-versatic acid.

36. The method according to claim 30, wherein the total amount of component (b) and component (c) combined is 8 to 30% by weight.

37. The method according to claim 30, wherein the at least one additional free-radically copolymerizable monomer is present in an amount up to 5% by weight.

38. The method according to claim 30, wherein the % by weight content of components (a), (b) and (c) totals 100%.

39. The method according to claim 30, wherein the copolymer is prepared by free-radically initiated polymerization of components (a), (b), (c) and (d).

40. The method according to claim 30, wherein the substance comprises at least one selected from the group consisting of biologically active substances, pharmaceutically active substances, cosmetic ingredients, agrochemical ingredients, food supplements, dietetic agents, foods, food additives, dyes, and combinations thereof.

41. A preparation comprising:
a substance selected from the group consisting of substances which are insoluble in water, substances which are only sparingly soluble in water, and combinations thereof; and
a solubilizing amount of a copolymer; wherein the copolymer comprises:
(a) 60 to 99% by weight of at least one monomer selected from the group consisting of N-vinyllactams, N-vinylamides, and mixtures thereof;
(b) 1 to 40% by weight of at least one monomer selected from the group consisting of vinyl esters of aliphatic branched C₈-C₃₀-carboxylic acids;
(c) 0 to 30% by weight of vinyl acetate; and
(d) 0 to 39% by weight of at least one additional free-radically copolymerizable monomer;
wherein the % by weight content of components (a), (b), (c) and (d) totals 100%, and with the proviso that the total amount of component (b) and component (c) combined is 1 to 40% by weight based on the copolymer.

42. The preparation according to claim 41, wherein the copolymer comprises:
(a) 70 to 95% by weight of the at least one monomer selected from the group consisting of N-vinyllactams, N-vinylamides, and mixtures thereof;
(b) 5 to 30% by weight of the at least one monomer selected from the group consisting of vinyl esters of aliphatic branched C₈-C₃₀-carboxylic acids;
(c) 0 to 25% by weight of vinyl acetate; and
(d) 0 to 25% by weight of the at least one additional free-radically copolymerizable monomer.

43. The preparation according to claim 41, wherein the copolymer has a K value of 10 to 200.

44. The preparation according to claim 41, wherein the at least one monomer selected from the group consisting of N-vinyllactams, N-vinylamides, and mixtures thereof comprises a compound selected from the group consisting of N-vinylpyrrolidone and N-vinylcaprolactam.

45. The preparation according to claim 41, wherein the at least one monomer selected from the group consisting of vinyl esters of aliphatic branched C₈-C₃₀-carboxylic acids comprises a vinyl ester of a versatic acid.

46. The preparation according to claim 41, wherein the at least one monomer selected from the group consisting of vinyl esters of aliphatic branched C₈-C₃₀-carboxylic acids comprises a vinyl ester of a C₉-C₁₀-versatic acid.

47. The preparation according to claim 41, wherein the total amount of component (b) and component (c) combined is 8 to 30% by weight.

48. The preparation according to claim 41, wherein the % by weight content of components (a), (b) and (c) totals 100%.

49. The preparation according to claim 41, wherein the substance comprises at least one selected from the group consisting of biologically active substances, pharmaceutically active substances, cosmetic ingredients, agrochemical ingredients, food supplements, dietetic agents, food additives, dyes, and combinations thereof.
